(19) United States
(12) Patent Application Publication
(10) Pub. No.: US 2025/0107910 A1
(43) Pub. Date: Apr. 3, 2025

(54) DELIVERY DEVICE

(71) Applicant: OLYMPUS MEDICAL SYSTEMS ORP., Tokyo (JP)

(72) Inventor: Yoichi SAKURADA, Higashimurayama-shi (JP)

(21) Appl. No.: 18/978,860

(22) Filed: Dec. 12, 2024

Related U.S. Application Data
(63) Continuation of application No. PCT/JP2022/029158, filed on Jul. 28, 2022.

(51) Int. Cl. A61F 2/95; A61F 2/966

(52) U.S. Cl. CPC A61F 2/9517; A61F 2/966

(57) ABSTRACT

A delivery device includes: an outer cylinder; an inner cylinder which is configured to be inserted through the outer cylinder; a shaft member that is a flexible elongated member and is configured to be inserted through the inner cylinder to hold a stent between a distal end portion and the inner cylinder; an operation mechanism which includes a housing and is operated to relatively move the inner cylinder relative to the shaft member in a longitudinal direction; and an adjuster which is provided from a proximal end of the outer cylinder to a proximal end of the shaft member and is configured to correct the relative position of the outer cylinder relative to at least one of the inner cylinder or the shaft member in the longitudinal direction.

### CROSS-REFERENCE TO RELATED APPLICATIONS

The present application is a continuation application based on PCT Patent Application No. PCT/JP2022/029158, filed on Jul. 28, 2022, the entire content of which is hereby incorporated by reference.

### TECHNICAL FIELD

The present invention relates to a delivery device.

### BACKGROUND ART

A stent delivery device is used to place a self-expanding stent in a body cavity (for example, U.S. Pat. No. 5,733,267, which is referred to as Patent Document 1). In the conventional stent delivery device, the stent is accommodated in a gap between an inner sheath and an outer sheath. The outer sheath is retracted relative to the inner sheath to expose the stent and expand its diameter, and then the inner sheath is removed from the stent to leave the stent in the body cavity. The stent delivery device is inserted into a forceps port through an endoscope channel of an endoscope insertion section of an endoscope inserted into the body cavity and used. Since the endoscope insertion section meanders inside the body, the sheath of the stent delivery device also meanders relative to the endoscope channel. Further, since the portion of the stent delivery device exposed outside the forceps port does not cause frictional resistance like the portion inserted into the endoscope channel, that portion is easily deformed by an external force. Therefore, in the stent delivery device, the portion exposed outside the forceps port of the endoscope is easily straightened. In the conventional stent delivery device, when the outer sheath is returned toward a hand to place the stent inside the body, the relative position between the inner sheath and the outer sheath may be displaced from the expected range due to the meandering of the sheath inside the endoscope channel or straightening of the portion exposed outside the forceps port. Since high-accuracy positioning is required at the time of placing the stent in a treatment site, it takes time for fine adjustment of the stent to accurately place the stent if unexpected positional displacement occurs in the stent delivery device.

The delivery system described in Patent Document 1 has a triple structure including an inner shaft that holds a stent, an intermediate shaft, and a reinforcing outer shaft. The delivery system having a triple structure can reduce the above-described unexpected positional displacement of the inner shaft at the time of placing the stent compared to a conventional delivery system having a double structure. However, in the delivery system described in Patent Document 1, since the intermediate shaft is provided between the reinforcing outer shaft and the inner shaft, which are fixed via a manifold, to be capable of advancing and retracting, the intermediate shaft is displaced relative to the outer shaft. As a result, the delivery system described in Patent Document 1 still has difficulty in accurately placing the stent, and cannot shorten the procedure time. In addition, a large force is required to pull the intermediate shaft due to friction between the intermediate sheath and the outer sheath.

### SUMMARY

The present invention provides a delivery device that is reliably placed at a desired position.

A delivery device according to an aspect of the present invention includes: an outer cylinder; an inner cylinder which is configured to be inserted through the outer cylinder; a shaft member that is a flexible elongated member and is configured to be inserted through the inner cylinder to hold a stent between a distal end portion and the inner cylinder; an operation mechanism which includes a housing and is operated to relatively move the inner cylinder relative to the shaft member in a longitudinal direction; and an adjuster which is provided from a proximal end of the outer cylinder to a proximal end of the shaft member and is configured to correct the relative position of the outer cylinder relative to at least one of the inner cylinder or the shaft member in the longitudinal direction.

A delivery device according to an aspect of the present invention includes: an outer cylinder; an inner cylinder which is configured to be inserted through the outer cylinder; a shaft member that is a flexible elongated member and is configured to be inserted through the inner cylinder to hold a stent between a distal end portion and the inner cylinder; an operation mechanism which includes a housing and is operated to relatively move the inner cylinder relative to the shaft member in a longitudinal direction; and an adjuster which is provided from a proximal end of the outer cylinder to a proximal end of the shaft member and is configured to correct the relative position between a proximal end of the outer cylinder and a proximal end of the shaft member in the longitudinal direction.

According to the above aspect, the stent can be positioned and placed with high accuracy at a desired position at the time of placing the stent.

### BRIEF DESCRIPTION OF DRAWINGS

FIG. 1 is a diagram showing an endoscope system according to a first embodiment.

FIG. 2 is a cross-sectional view showing an overall configuration of a delivery device according to the first embodiment.

FIG. 3 is a cross-sectional view showing an overall configuration of the delivery device according to the first embodiment.

FIG. 4 is a diagram showing a usage mode of the delivery device according to the first embodiment.

FIG. 5 is a diagram showing a usage mode of the delivery device according to the first embodiment.

FIG. 6 is a perspective view showing a modified example of an operation section of the delivery device.

FIG. 7 is a perspective view showing a modified example of the operation section of the delivery device.

FIG. 8 is a perspective view showing a modified example of the operation section of the delivery device.

FIG. 9 is a cross-sectional view showing an overall configuration of a delivery device of an endoscope system according to a second embodiment.

FIG. 10 is a cross-sectional view showing an overall configuration of the delivery device of the endoscope system according to the second embodiment.

FIG. 11 is a cross-sectional view showing an overall configuration of a delivery device of an endoscope system according to a third embodiment.

FIG. 12 is a cross-sectional view showing an overall configuration of a delivery device of an endoscope system according to a fourth embodiment.

FIG. 13 is a cross-sectional view showing an overall configuration of a delivery device according to a fifth embodiment.

FIG. 14 is a schematic view illustrating the delivery device according to the first embodiment.

FIG. 15 is a schematic view illustrating the delivery device according to the first embodiment.

### DESCRIPTION OF EMBODIMENTS

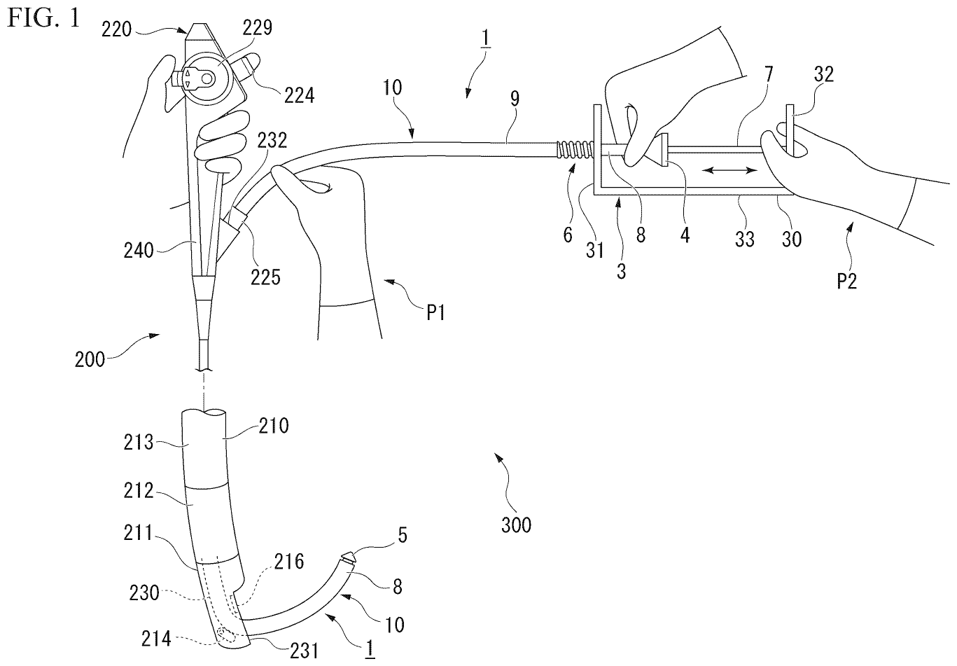

Embodiments of the present invention will be described with reference to FIGS. 1 to 5.

### First Embodiment

[Endoscope System 300] An endoscope system 300 will be described with reference to FIG. 1. The endoscope system 300 includes an endoscope 200 and a delivery device 1. The delivery device 1 is inserted through a treatment tool channel 230 of the endoscope 200.

### [Endoscope 200]

The endoscope 200 will be described with reference to FIG. 1.

The endoscope 200 is a known side-viewing flexible endoscope, and includes an elongated insertion section 210, an operation section 220, and a treatment tool channel 230. The operation section 220 is provided at the proximal end of the insertion section 210. In the following description, the side of the operation section 220 of the endoscope 200 is referred to as a proximal direction L2. The opposite side of the operation section 220 in the longitudinal direction of the insertion section 210 is referred to as a distal direction L1 of the endoscope 200. The treatment tool channel 230 is a channel through which a treatment tool of the delivery device 1 is inserted. The endoscope 200 may be a direct-viewing type flexible endoscope.

The insertion section 210 includes a distal end rigid portion 211, a bending portion 212, and a flexible cylinder portion 213. The distal end rigid portion 211 is provided at the distal end of the insertion section 210. The bending portion 212 is attached to the proximal side of the distal end rigid portion 211 and is configured to be bendable. The flexible cylinder portion 213 is attached to the proximal side of the bending portion 212.

An imaging unit 216 is provided in an exposed state to the outside on a side surface of the distal end rigid portion 211. The imaging unit 216 includes a light guide and a CCD.

The distal end rigid portion 211 is provided with a lifting base 214. The proximal end of the lifting base 214 is rotatably supported by the distal end rigid portion 211. A lifting base operation wire (not shown) is fixed to the distal end of the lifting base 214. The lifting base operation wire (not shown) extends through the insertion section 210 in the proximal direction L2.

The bending portion 212 is configured to be bendable in the vertical direction or the horizontal direction. In the bending portion 212, the distal end of the operation wire is fixed to the distal side of the bending portion 212. The operation wire extends to the operation section 220 through the insertion section 210. The vertical direction is the vertical direction of the field of view of the endoscope among the orthogonal directions in which the insertion section 210 is bent in a direction intersecting the axis from a straight extension state. The horizontal direction is the horizontal direction of the field of view of the endoscope among the orthogonal directions in which the insertion section 210 is bent in a direction intersecting the axis from a straight extension state. The bending direction of the bending portion 212 is not limited to the vertical direction and the horizontal direction, but the bending portion is also bendable in a direction intersecting the axis of the insertion section 210.

The distal end portion of the treatment tool channel 230 opens to the side surface of the distal end rigid portion 211. The proximal end of the treatment tool channel 230 extends to the operation section 220.

The proximal end of the operation section 220 is provided with a knob 229 which operates an operation wire or a switch 224 which operates the imaging unit 216 and the like. A user can bend the bending portion 212 in a desired direction by operating the knob 229. The operation section 220 is not limited to the above-described example as long as the operation section is configured to operate the operation wire or the imaging unit 216.

A forceps port 232 which communicates with the treatment tool channel 230 is provided on the distal side of the operation section 220. The user can insert an endoscopic treatment tool such as the delivery device 1 from the forceps port 232. A forceps plug 225 is attached to the forceps port 232 to prevent leakage of body fluids.

### [Delivery Device 1]

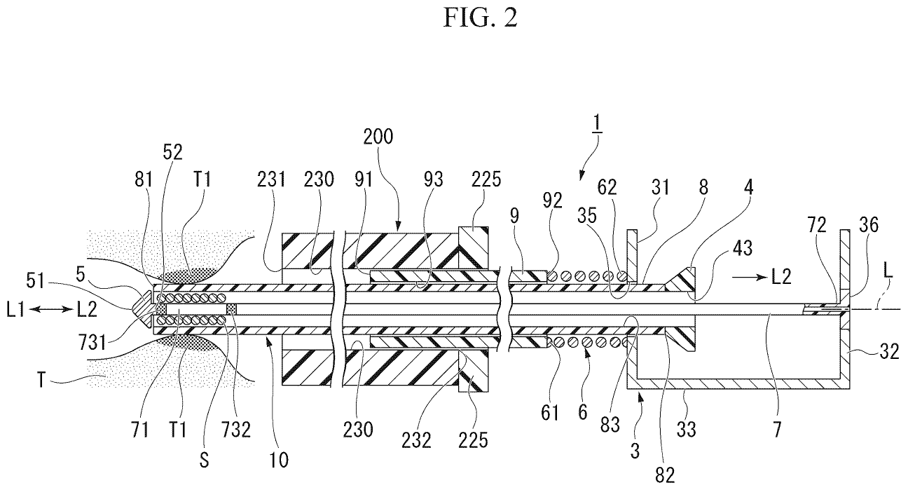

The delivery device 1 will be described with reference to FIGS. 2 to 5. The delivery device 1 is a treatment tool that holds a stent S at the distal end portion and places the stent S in the body via the endoscope 200. As shown in FIG. 2, the delivery device 1 has an overall elongated shape. The delivery device 1 includes an outer cylinder 9, an inner cylinder 8, a shaft member 7, an adjuster 6, and an operation section 3 (operation mechanism). In the delivery device 1, the inner cylinder 8 and the shaft member 7 are inserted into the outer cylinder 9 to be capable of advancing and retracting. The operation section 3 and the adjuster 6 are provided at the proximal portion of the delivery device 1. The shaft member 7, the inner cylinder 8, and the outer cylinder 9 are referred to as a treatment tool body 10.

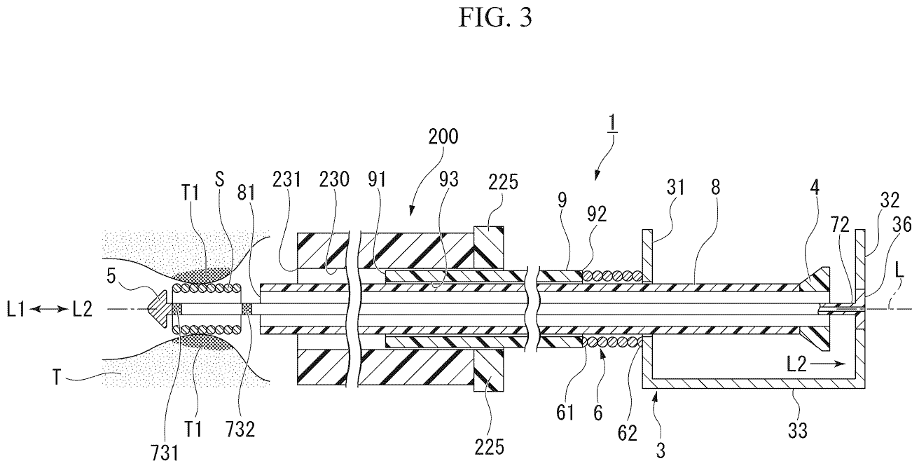

The outer cylinder 9 is a flexible elongated cylindrical member. The outer cylinder 9 may be a tube made of resin or the like, or may be a coil sheath. The length of the outer cylinder 9 is shorter than the lengths of the inner cylinder 8 and the shaft member 7. As shown in FIGS. 2 and 3, the outer cylinder 9 includes a lumen (internal space) 93 formed in the longitudinal direction L from a distal end 91 to a proximal end 92, and is open at the distal end 91 and the proximal end 92. The lumen 93, the distal end 91, and the proximal end 92 are opened in a substantially circular shape so that the inner cylinder 8 can be inserted therethrough. The outer cylinder 9 may have any length that enables the distal end 91 of the outer cylinder 9 to be disposed on the distal side of the forceps port 232 when the delivery device 1 is inserted into the treatment tool channel 230 of the endoscope 200.

The inner cylinder 8 is a flexible elongated cylindrical member. The inner cylinder 8 may be a tube made of resin or the like, or may be a coil sheath. As shown in FIGS. 2 and 3, a lumen (internal space) 83 is formed in the longitudinal direction L from a distal end 81 to a proximal end 82 of the inner cylinder 8, and is opened at the distal end 81 and the proximal end 82. The lumen 83, the distal end 81, and the proximal end 82 are opened in a substantially circular shape so that the shaft member 7 and the contracted stent S can be inserted therethrough. The lumen 83, the distal end 81, and the proximal end 82 may further have an opening large enough to allow a guide wire (not shown) to be inserted therethrough. As shown in FIGS. 1 and 2, a handle 4 is provided at the proximal end 82 of the inner cylinder 8. As shown in FIGS. 2 and 3, the handle 4 includes an insertion passage 43 which communicates with the lumen 83 of the inner cylinder 8.

The shaft member 7 is a flexible elongated member that is insertable through the lumen 83 of the inner cylinder 8. The shaft member 7 is configured to be able to hold the stent S between the shaft member and the inner cylinder 8. The shaft member 7 is, for example, a wire made of metal such as NiTi or resin. A tip 5 is fixed to the distal end of the shaft member 7. In the case of the delivery device used under X-ray fluoroscopy, the distal end portion of the shaft member 7 may be provided with X-ray-opaque metallic markers 731 and 732. At the time of placing the stent S, the metallic markers 731 and 732 indicate the storage positions of the distal end and the proximal end of the stent S within the body cavity under X-ray fluoroscopy.

The tip 5 is provided at a distal end 71 of the shaft member 7. The diameter of the tip 5 is larger than the inner diameter of the lumen 83 of the inner cylinder 8 and is equal to or smaller than the outer diameter of the inner cylinder 8. The tip 5 comes into contact with the distal end 81 of the inner cylinder 8 at the position where the shaft member 7 is most retracted. The tip 5 is provided to prevent the stent S accommodated in the inner cylinder 8 from falling off and to facilitate confirmation of the distal end position of the shaft member 7 through an endoscopic image. The tip 5 includes a substantially conical distal end portion 51 that protrudes toward the distal side. As shown in FIGS. 2 and 3, the tip 5 is connected to the shaft member 7 at the proximal end 52. The distal end portion 51 has a smaller diameter dimension than the proximal end 52. Although not shown in the drawings, a through-hole is formed in the tip 5 so that a guide wire inserted through the inner cylinder 8 can be inserted therethrough.

The delivery device 1 includes the inner cylinder 8 and the shaft member 7 provided along the entire length. The length of the shaft member 7 is longer than the inner cylinder 8. As shown in FIGS. 2 and 3, the shaft member 7 is inserted through the lumen 83 of the inner cylinder 8. Specifically, the shaft member 7 is inserted through the lumen 83 and is passed through the openings of the distal end 81 and the proximal end 82 of the inner cylinder 8 to be movable relative to the inner cylinder 8. The tip 5 is disposed to protrude toward the distal side of the distal end 81 of the inner cylinder 8.

The operation section 3 is provided at the proximal portion of the delivery device 1. The operation section 3 performs an operation of advancing and retracting the inner cylinder 8 relative to the outer cylinder 9 and the shaft member 7, an operation of advancing and retracting the delivery device 1 inside the treatment tool channel, an operation of bending the outer cylinder 9, the inner cylinder 8, and the shaft member 7, and the like. The operation section 3 includes a housing 30 and the handle 4. The housing 30 is a portion of the delivery device 1 that is gripped by a surgeon P2. The housing 30 includes a distal portion 31, a proximal portion 32, and an intermediate portion 33. The distal portion 31 and the proximal portion 32 have a flat plate shape that is substantially perpendicular to the longitudinal direction L, and are disposed parallel to each other and spaced apart. The intermediate portion 33 extends in the longitudinal direction L and connects the distal portion 31 and the proximal portion 32 to each other. The proximal portion 32 is provided with a fixing portion 36 of a proximal end 72 of the shaft member 7. In the distal portion 31, an opening portion 35 is formed on the distal side of the fixing portion 36. The opening portion 35 is an opening which penetrates the distal portion 31 in the longitudinal direction L and has a size in which the inner cylinder 8 is capable of advancing and retracting.

The proximal region of the inner cylinder 8 and the shaft member 7 is disposed in the housing 30 of the operation section 3. The inner cylinder 8 and the shaft member 7 are inserted through the opening portion 35 of the housing 30. The proximal end of the shaft member 7 extends in the longitudinal direction L on the proximal side of the proximal end 82 of the inner cylinder 8. The proximal end 72 of the shaft member 7 is fixed to the fixing portion 36.

The proximal end 82 of the inner cylinder 8 and the handle 4 are provided inside the housing 30. The handle 4 is disposed between the distal portion 31 and the proximal portion 32 of the housing 30. When the handle 4 is operated to advance and retract inside the housing 30, the proximal end 82 of the inner cylinder 8 advances and retracts relative to the shaft member 7.

The outer cylinder 9 is provided on the distal side of the operation section 3. The adjuster 6 is provided between the proximal end 92 of the outer cylinder 9 and the operation section 3. The adjuster 6 can adjust the relative position of the outer cylinder 9 in the longitudinal direction L relative to any one of the inner cylinder 8 and the shaft member 7. The adjuster 6 can adjust the relative position between the proximal end 92 of the outer cylinder 9 and the proximal end 72 of the shaft member 7 in the longitudinal direction L.

The adjuster 6 is, for example, a coil spring which is disposed coaxially with the outer cylinder 9. A specific example of the adjuster 6 is a compression spring. The adjuster 6 contracts in the longitudinal direction L by receiving a force in the proximal direction L2 from the outer cylinder 9. The adjuster 6 is expandable and contractible between a reference length in the longitudinal direction L of the adjuster 6 when the outer cylinder 9 has a linear shape and no load is applied and a correction length which becomes shorter than the reference length due to an external force. The adjuster 6 may be configured to include an elastic member that is contractible in the longitudinal direction L when an external force is applied.

The end of the adjuster 6 is fixed to the distal portion 31 of the housing 30 or the proximal end 92 of the outer cylinder 9. Both ends of the adjuster 6 in the longitudinal direction L may be fixed to both the distal portion 31 of the housing 30 and the proximal end 92 of the outer cylinder 9. Alternatively, both ends of the adjuster 6 in the longitudinal direction L may be disposed to come into contact with the distal portion 31 of the housing 30 and the proximal end 92 of the outer cylinder 9.

The stent S is a cylindrical self-expanding stent. The stent S is formed by braiding wires. The stent S has a cylindrical shape extending in the longitudinal direction L. The wire forming the stent S is a superelastic alloy based on NiTi. The superelastic alloy based on is NiTi is not permanently deformed when the alloy is woven, and the woven shape is memorized by applying heat treatment to the alloy in a woven state. The stent S is capable of shrinking from its natural state when an external force is applied, and has sufficient strength to hold the lumen without blocking the narrowed portion.

An example in which the stent S is a self-expanding stent has been described, but the stent S is not limited to the self-expanding stent. The stent S may be a non-self-expanding stent, and examples thereof include a CoCr-based alloy stent and a biodegradable stent made of polylactic acid, polyglycolic acid, or a copolymer thereof. The stent S may be a fluid-expandable stent. Example of the fluid-expandable stent is a non-self-expanding stent that is expanded by other treatment tools such as a balloon.

As shown in FIG. 2, the stent S is accommodated in the lumen 83 of the distal end 81 of the inner cylinder 8. Specifically, the shaft member 7 is inserted through the stent S, and the contracted stent S is accommodated in a gap between the inner cylinder 8 and the shaft member 7. The stent S is engaged to an engaging portion (not shown) formed on the outer peripheral surface of the shaft member 7. Accordingly, the stent S is positioned to the shaft member 7 in the contracted state, and does not move relatively in the longitudinal direction L of the shaft member 7. When the handle 4 is pulled in the proximal direction L2, the inner cylinder 8 moves in the proximal direction L2, and the stent S expands to be released from the delivery device 1.

Next, the operation of the endoscope system 300 will be described.

In the delivery device 1, the stent S is fitted onto the distal end portion of the shaft member 7, and the stent S is held between the inner cylinder 8 and the shaft member 7. Specifically, the distal end 71 of the shaft member 7 is allowed to protrude toward the distal side of the distal end 81 of the inner cylinder 8, and the expanded stent S is inserted into the tip 5 and the distal end 71 of the shaft member 7. When the shaft member 7 is retracted after the stent S is disposed on the proximal side of the tip 5, the proximal end of the stent S is pressed between the shaft member 7 and the distal opening of the inner cylinder 8 by the distal end 81 of the inner cylinder 8, and the stent S gradually contracts. When the shaft member 7 is retracted until the tip 5 comes into contact with the distal end 81 of the inner cylinder 8, the stent S is accommodated between the inner cylinder 8 and the shaft member 7.

The delivery device 1 accommodating the stent S is inserted into the treatment tool channel 230 of the endoscope 200 inserted into the patient's body. The inner cylinder 8 and the shaft member 7 are inserted to a position where they protrude from a distal opening 231 of the treatment tool channel 230. The distal end 91 of the outer cylinder 9 is inserted through the treatment tool channel 230 of the endoscope 200 and is connected to the treatment tool channel 230. For example, the distal end portion of the outer cylinder 9 is crimped and connected to the forceps port 232. The connection position and method between the distal end 91 of the outer cylinder 9 and the treatment tool channel 230 are not limited to the above-described example. The distal end 91 of the outer cylinder 9 may be inserted to the distal side of the forceps port 232 to be connected to the endoscope 200. When the delivery device 1 is in use, the outer cylinder 9 suppresses the meandering of the portion of the treatment tool body 10 exposed to the proximal side of the forceps port 232 and suppresses a change in path length between the inner cylinder 8 and the shaft member 7.

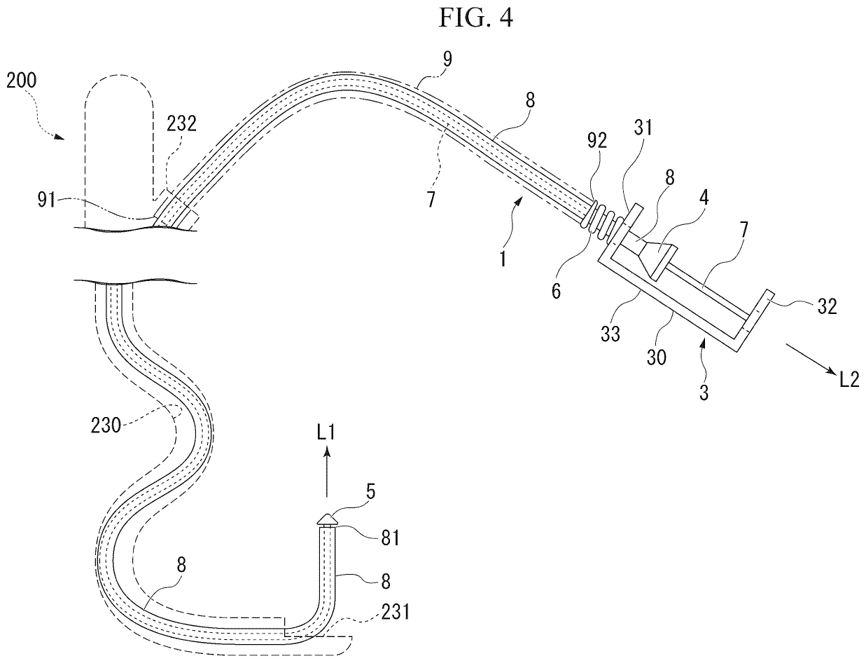

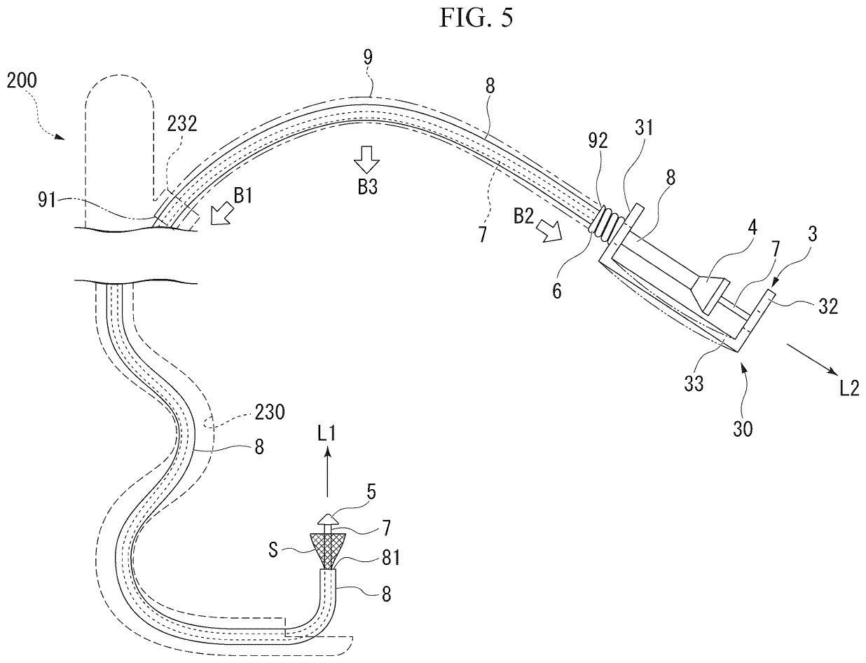

FIG. 4 schematically shows the path of the inner cylinder 8 and the shaft member 7 inside the treatment tool channel 230 and the path of the inner cylinder 8 and the shaft member 7 inside the outer cylinder 9 in the external exposed portion of the treatment tool body 10 when the treatment tool body 10 is operated to advance relative to the treatment tool channel 230. FIG. 5 schematically shows the path of the inner cylinder 8 and the shaft member 7 inside the treatment tool channel 230 and the path of the inner cylinder 8 and the shaft member 7 inside the outer cylinder 9 in the external exposed portion of the treatment tool body 10 when the stent is placed, that is, the inner cylinder 8 is operated to retract. As shown in FIGS. 4 and 5, since the delivery device 1 is inserted through the treatment tool channel 230 of the insertion section 210 of the endoscope 200 which is meandering in the body cavity, the shaft member 7 and the inner cylinder 8 also meander. When the shaft member 7 and the inner cylinder 8 meander, a change occurs in the path length of the shaft member 7 inside the lumen 83 of the inner cylinder 8. As a result, the distal end 81 of the inner cylinder 8 moves in the distal direction L1 against the surgeon's will. As shown in FIG. 4, when the treatment tool body 10 is operated to advance relative to the treatment tool channel 230, the handle 4 is disposed at the most advanced position, the distal end 81 of the inner cylinder 8 is disposed adjacent to the tip 5, and in this state, the entire housing 30 is advanced. The tip 5 is advanced to the vicinity of the narrow portion of the lumen. By maintaining the position of the housing 30 relative to the forceps port 232, the position of the treatment tool body 10 relative to the treatment tool channel 230 is maintained.

Next, the stent S is released. Specifically, when the handle 4 is retracted in the proximal direction L2 relative to the housing 30 as shown in FIG. 5 while maintaining the position of the housing 30 relative to the forceps port 232, the inner cylinder 8 retracts relative to the shaft member 7 while the position of the tip 5 is maintained in the vicinity of the narrow portion. As the inner cylinder 8 retracts, the stent S is gradually exposed and expanded due to the self-expanding function of the stent S. At this time, the static friction and dynamic friction of the stent S with the outer peripheral surface of the shaft member 7 at the reduced diameter portion is sufficiently larger than the dynamic friction of the stent S with the inner wall of the lumen 83 of the inner cylinder 8. Therefore, the stent S is maintained at the position disposed in the distal portion of the shaft member 7.

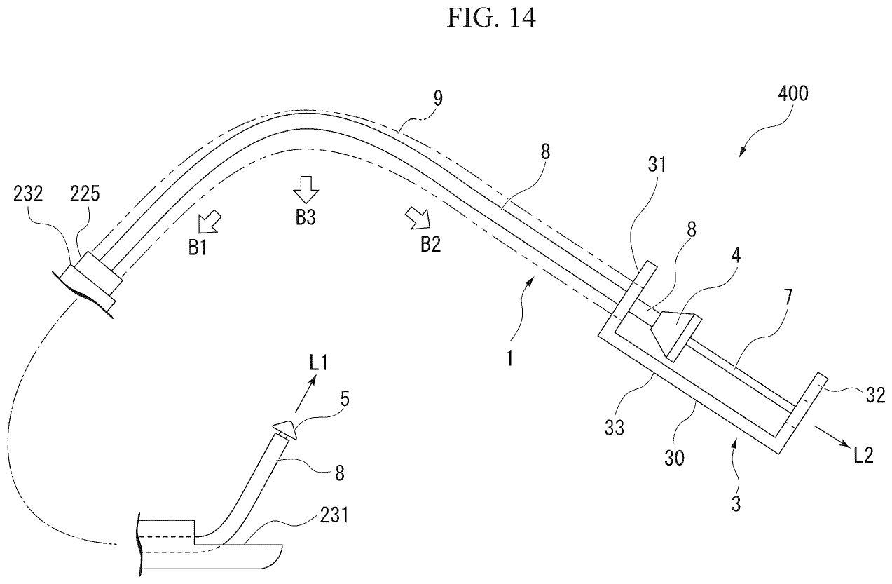

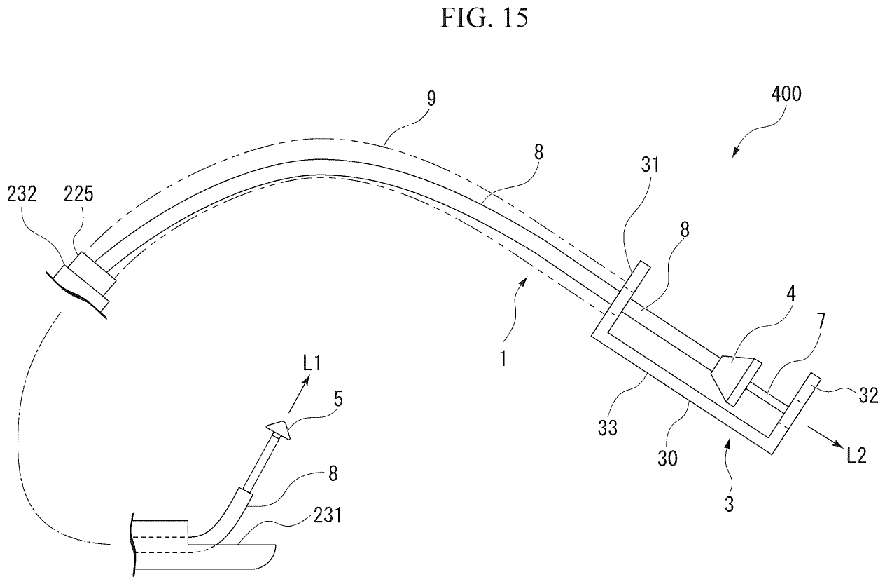

The treatment tool body 10 between the position (forceps port 232) where the surgeon grips the delivery device 1 and the housing 30 is bendable according to the position of the housing 30 and the like. The relative positions of the shaft member 7, the inner cylinder 8, and the outer cylinder 9 in the treatment tool body 10 are different when the treatment tool body 10 extends straight in the longitudinal direction L and when the treatment tool body 10 is bent. In the case of the straight line, the outer cylinder 9, the inner cylinder 8, and the shaft member 7 are arranged substantially coaxially, and the length (path length) of the portion disposed inside the outer cylinder 9 in the shaft member 7 and the inner cylinder 8 is substantially equal to the length of the outer cylinder 9. In contrast, when the treatment tool body 10 is bent significantly, the central axes of the outer cylinder 9, the shaft member 7, and the inner cylinder 8 are misaligned with each other, and the path lengths of the shaft member 7 and the inner cylinder 8 inside the outer cylinder 9 change. As shown in FIGS. 4 and 5, when the treatment tool body 10 is bent and the proximal end 82 of the inner cylinder 8 is pulled in the proximal direction L2 while the positions of the shaft member 7 and the outer cylinder 9 are maintained, the inner cylinder 8 is subjected to forces applied to the inside of the curve of the treatment tool body 10, that is, in the directions indicated by the arrows B1, B2, and B3 in FIG. 5. This force is applied to the outer cylinder 9 to linearly move the outer cylinder from the inner cylinder 8 in the direction of the arrow B2 inside the lumen 93. When a force is applied to the outer cylinder 9 in the direction indicated by the arrow B3 in FIG. 5 while the inner cylinder 8 inside the lumen 93 is in a position biased toward the outside of the curve of the outer cylinder 9, the inner cylinder 8 also straightens. As a result, the path length of the inner cylinder 8 which has been meandering inside the lumen 93 changes, and the relative length of the outer cylinder 9 relative to the inner cylinder 8 becomes longer. When the handle 4 is pulled in this state, a compressive force is applied to the outer cylinder 9 in the longitudinal direction L. Since the distal end 91 of the outer cylinder 9 is connected to the treatment tool channel 230, a compressive force in the longitudinal direction L of the outer cylinder 9 is applied to move the proximal end 92 in the proximal direction L2. Here, a comparison with the case where the adjuster 6 is not provided will be made with reference to FIGS. 14 and 15. FIGS. 14 and 15 are diagrams showing a case where an adjuster is not provided to compare the difference between the presence and absence of the adjuster. As shown in FIG. 14, when the treatment tool body 10 is bent significantly, the central axis of the inner cylinder 8 is misaligned inside the lumen 93. In this state, when a force for straightening the outer cylinder 9 is applied in the direction indicated by the arrow B3, the compressive force in the longitudinal direction L of the outer cylinder 9 moves the striking proximal end 82 in the proximal direction L2. When the adjuster 6 is not provided, the inner cylinder 8 advances relative to the outer cylinder 9 as the inner cylinder 8 is straightened, and the position of the tip 5 moves relatively to the distal side as shown in FIG. 15. Since the relative movement amount of the inner cylinder 8 toward the distal side caused by the straightening of the inner cylinder 8 is larger than the force that relatively moves the operation section 3 in the proximal direction L2, the position of the tip 5 will also be unintentionally displaced in the distal direction L1 when the adjuster is not provided. However, in the delivery device 1, since the adjuster 6 is provided at the proximal end 92 of the outer cylinder 9, a force in the proximal direction is applied to the adjuster 6 from the outer cylinder 9, and the adjuster 6 contracts in the longitudinal direction L. Since the adjuster 6 is configured to contract by the same amount as the length by which the inner cylinder 8 advances due to a change in path length, it is possible to correct the change in path length. Since the tip 5 is prevented from being displaced in the distal direction L1 as described above as a result of the adjuster 6 that corrects the change in path length, it is possible to prevent the position of the distal end 71 of the shaft member 7 from being displaced after a placement position T1 is set.

Further, as a result of the change in path length of the inner cylinder 8 inside the lumen 93 of the outer cylinder 9, the number of points where the inner cylinder 8 contacts the inner wall of the lumen 93 of the outer cylinder 9 increases. As a result, the frictional force between the inner cylinder 8 and the outer cylinder 9 increases, and a very large pulling force is required when pulling the handle 4. However, since the adjuster 6 is provided at the proximal end 92 of the outer cylinder 9, a force in the proximal direction L2 is applied to the adjuster 6 from the outer cylinder 9 when a force that changes the path length of the inner cylinder 8 inside the lumen 93 is applied. Accordingly, since the adjuster 6 contracts in the longitudinal direction L, the change in path length can be corrected. As a result of correcting the change in path length, the number of contact points between the inner cylinder 8 and the outer cylinder 9 decreases. Accordingly, since an increase in frictional force is prevented, it is possible to suppress an increase in the force of pulling the handle 4 required at the time of placing the stent S. As a result, the procedure can be performed smoothly and the procedure time can be prevented from being prolonged.

In addition, when the adjuster 6 is not provided, a compressive force in the longitudinal direction L of the outer cylinder 9 is applied to the housing 30, and the intermediate portion 33 of the housing 30 may be bent as indicated by the imaginary line in FIG. 5. Similarly, when the adjuster 6 is not provided, a large force is required when pulling the handle 4 in accordance with an increase in frictional force, and the intermediate portion 33 of the housing 30 may be bent as indicated by the imaginary line in FIG. 5. When the intermediate portion 33 is bent, the relative length between the shaft member 7 and the inner cylinder 8 changes slightly. Since the placement position T1 of the stent S is a very small location and the stent S is also small, high positioning accuracy is required at the time of placing the stent S. Therefore, even when the change in relative length caused by the bending of the housing 30 is small, the influence on the positioning of the placement position T1 is large. However, since the adjuster 6 is provided in the delivery device 1, it is possible to prevent deformation of the housing 30 due to an external force and to prevent a decrease in the positioning accuracy when the stent S is placed.

Furthermore, when the placement position T1 of the stent S is readjusted at a stage before the stent S is completely exposed from the distal end 81 of the inner cylinder 8, a recapture operation can be performed. Specifically, the inner cylinder 8 is advanced again while the position of the delivery device 1 relative to the insertion section 210 of the endoscope 200 is maintained, so that the distal end 81 of the inner cylinder 8 is advanced relative to the shaft member 7 and the stent S is accommodated in a stent accommodation region again. The surgeon advances the operation section 3 in the distal direction L1 while holding the housing 30. The shaft member 7 and the inner cylinder 8 advance from the distal opening 231, the stent S is accommodated in the inner cylinder 8, and the recapture operation is completed. When the recapture operation is completed, the state returns to that before the stent was released.

As shown in FIG. 3, when the stent S is completely exposed from the distal end 81 of the inner cylinder 8, the stent S expands to be larger than the diameter of the tip 5, contacts the inner wall of the lumen of the portion where the stent S is placed, and expands the narrowed portion. When the operation section 3 is retracted in this state, the tip 5 is passed through the stent S and is displaced in the proximal direction L2, and the stent S is placed. After the stent S is placed, the operation section 3 is pulled in the proximal direction L2, and the delivery device 1 is removed from the treatment tool channel 230.

According to the delivery device 1 and the endoscope system, the adjuster 6 can correct the change in path length of the inner cylinder 8 and the shaft member 7 inside the lumen 93 of the outer cylinder 9. Thus, since it is possible to correct the change in path length by the adjuster 6 even in the case of a tripe-structure treatment tool body 10 including the outer cylinder 9 that suppresses large bending of the inner cylinder 8 and the shaft member 7, it is possible to prevent the positional displacement of the shaft member 7 caused by the change in path length when the handle 4 is pulled at the time of placing the stent S. Therefore, the operation of placing the stent S can be performed smoothly.

According to the delivery device 1 and the endoscope system 300, the adjuster 6 can correct the relative position between the proximal end 92 of the outer cylinder 9 and the proximal end 72 of the shaft member 7 in the longitudinal direction L. Thus, since it is possible to correct the change in path length by the adjuster 6 even in the case of a tripe-structure treatment tool body 10 including the outer cylinder 9 that suppresses large bending of the inner cylinder 8 and the shaft member 7, it is possible to prevent the positional displacement of the shaft member 7 caused by the change in path length when the handle 4 is pulled at the time of placing the stent S. Therefore, the operation of placing the stent S can be performed smoothly.

According to the delivery device 1 and the endoscope system 300 of the above-described embodiment, the stent S can be positioned at a desired position with high accuracy at the time of placing the stent.

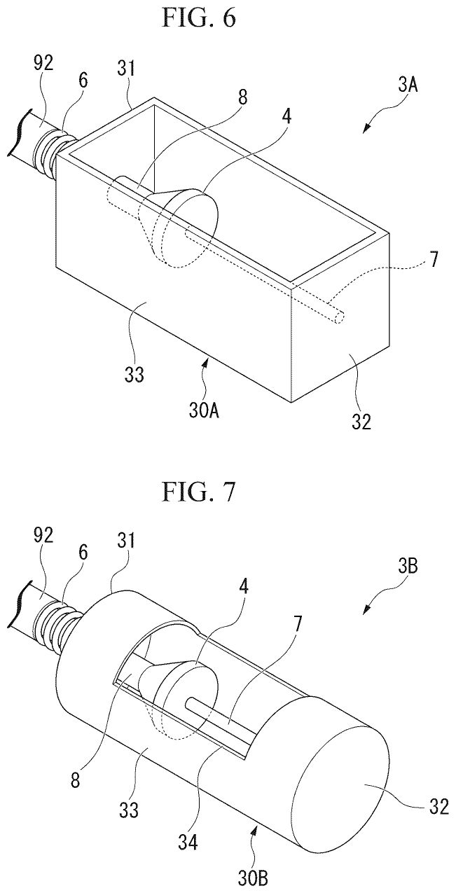

The configuration of the operation section 3 is not limited to the example shown in the above-described embodiment. FIGS. 6 to 8 show a modified example of the operation section. For example, as shown in FIG. 6, an operation section 3A may include a rectangular housing 30A in which a distal portion 31 and a proximal portion 32 are connected by a pair of intermediate portions 33. In the case of the rectangular housing 30A having a pair of intermediate portions 33, the strength of the housing 30A can be improved, and the bending of the intermediate portions 33 described above can be more effectively suppressed.

For example, as shown in FIG. 7, an operation section 3B may include a substantially cylindrical housing 30B with a side hole 34 opening on the side, and the handle 4 may be operated through the side hole 34. In the case of the cylindrical housing 30B, since the intermediate portion 33 is less likely to be deformed in the thickness direction (radial direction), the bending of the intermediate portion 33 due to the compressive force applied from the outer cylinder 9 can be more effectively suppressed. By configuring the housing 30 to have the side hole 34 opening therein like the operation section 3B, the area of the intermediate portion 33 can be made large, and the rigidity of the intermediate portion 33 can be increased.

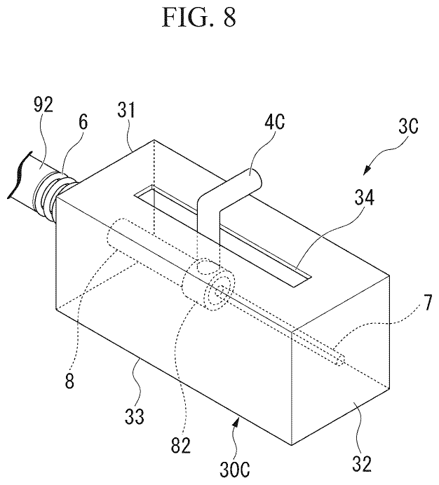

For example, as shown in FIG. 8, an operation section 3C may include a cubic housing 30C in which the intermediate portion 33 is formed by four side walls, and the side hole 34 may open. The side hole 34 of the housing 30C has a long slit shape along the longitudinal direction L. A handle 4C is fixed to the proximal end 82 of the inner cylinder 8. The handle 4C extends from the proximal end 82 in the direction (radial direction) orthogonal to the central axis of the inner cylinder 8 and protrudes from the side hole 34. As shown in the drawing, the handle 4C may have a hook shape extending in a direction perpendicular to the radial direction. In addition, the handle 4C may have a straight rod shape extending from the proximal end 82 in a direction (radial direction) orthogonal to the central axis of the inner cylinder 8 and protruding from the side hole 34. The handle 4C is slightly smaller than the opening of the side hole 34 and is provided to be movable along the side hole 34. Since the operation section 3C has a cubic shape and the area of the intermediate portion 33 can be made large, the rigidity of the intermediate portion 33 can be increased.

### Second Embodiment

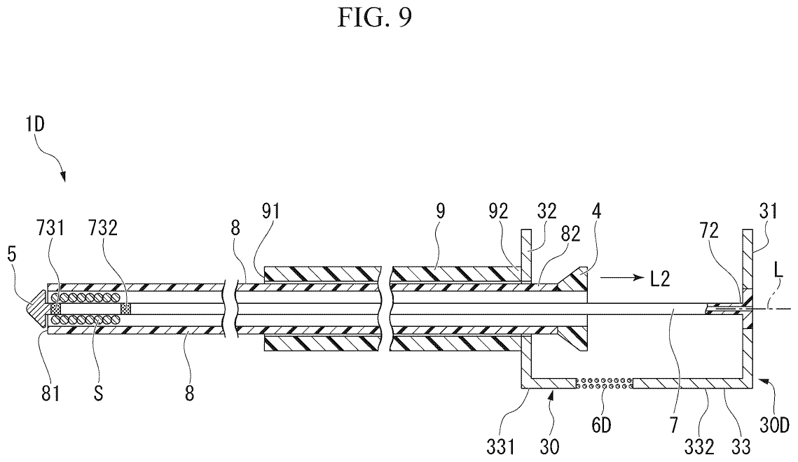

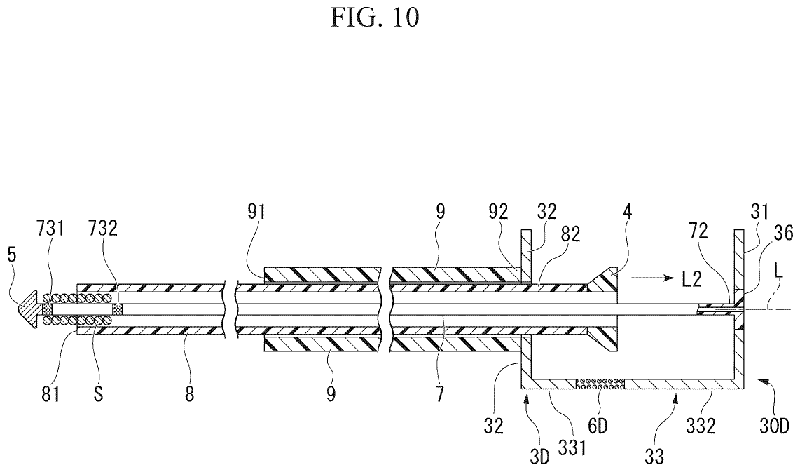

A delivery device 1D according to a second embodiment will be described with reference to FIGS. 9 and 10. In the following description, components common to those already described will be given the same reference numerals and duplicated description will be omitted. FIGS. 9 and 10 are cross-sectional views of the delivery device 1D according to this embodiment. The delivery device 1D is an example in which the configuration of an adjuster 6D is different from that of the first embodiment.

As shown in FIGS. 9 and 10, the adjuster 6D is provided in an operation section 3D. The adjuster 6D is provided in the intermediate portion 33 of the housing 30D. The intermediate portion 33 includes a distal wall 331 and a proximal wall 332 arranged side by side in the longitudinal direction L, and the adjuster 6D is provided between the distal wall 331 and the proximal wall 332. The adjuster 6D is a coil spring. The adjuster 6 is, for example, a compression spring. The adjuster 6D can expand and contract the length in the longitudinal direction L of the housing 30D. The proximal end 92 of the outer cylinder 9 is fixed to the distal portion 31 of the housing 30D.

If a force is applied from the outer cylinder 9 in the proximal direction L2 when the treatment tool body 10 is bent, the adjuster 6D contracts in the longitudinal direction L. When the bending of the treatment tool body 10 is eliminated, the adjuster 6D returns to the initial state. As shown in FIG. 5, at the time of placing the stent S, the treatment tool body 10 on the proximal side of the forceps port 232 is bent, and the handle 4 is pulled in the proximal direction L2 while the inner cylinder 8 is biased to the outside of the curve within the lumen 93. As a result of the force that straightens the outer cylinder 9, a compressive force is generated in the longitudinal direction L of the outer cylinder 9. This compressive force of the outer cylinder 9 exerts a force on the housing 30D in the proximal direction L2 to contract the adjuster 6D. The length of the intermediate portion 33 in the longitudinal direction L is temporarily shortened by the contraction of the adjuster 6. That is, the adjuster 6D corrects the force received from the outer cylinder 9. When the length of the intermediate portion 33 of the housing 30D in the longitudinal direction L is shortened, the distance between the proximal end 82 of the inner cylinder 8 and the proximal end 72 of the shaft member 7 becomes shorter, and the relative position between the inner cylinder 8 and the shaft member 7 changes. However, since the adjuster 6D can offset the compressive force of the outer cylinder 9, the change in path length of the inner cylinder 8, the shaft member 7, and the outer cylinder 9 can be corrected.

According to the delivery device 1D and the endoscope system 300, the adjuster 6D can correct the relative position between the proximal end 92 of the outer cylinder 9 and the proximal end 72 of the shaft member 7 in the longitudinal direction L. Thus, since it is possible to correct the change in path length by including the adjuster 6D even in the case of a tripe-structure treatment tool body 10 including the outer cylinder 9 that suppresses large bending of the inner cylinder 8 and the shaft member 7, it is possible to prevent the positional displacement of the shaft member 7 caused by the change in path length when the handle 4 is pulled at the time of placing the stent S. Therefore, the operation of placing the stent S can be performed smoothly.

### Third Embodiment

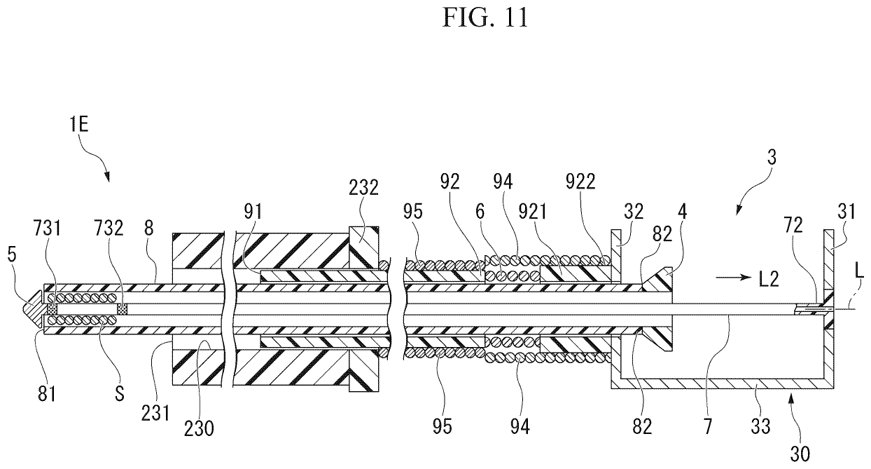

A delivery device 1E according to a third embodiment will be described with reference to FIG. 11. FIG. 11 is a cross-sectional view of a delivery device 1E according to this embodiment. The delivery device 1E includes the adjuster 6 and a reinforcing portion 921 adjacent to the proximal end 92 of the outer cylinder 9 in the proximal direction L2. The adjuster 6 is a coil spring similar to the adjuster 6 of the first embodiment. The reinforcing portion 921 is a tube thicker than the outer cylinder 9. The adjuster 6 and the reinforcing portion 921 are disposed coaxially with the outer cylinder 9. The opening diameter of the lumen of the reinforcing portion 921 is substantially the same as the opening diameters of the outer cylinder 9 and the adjuster 6. The proximal end 922 of the reinforcing portion 921 is fixed to the housing 30.

A first outer sheath 95 and a second outer sheath 94 are coil sheaths. The first outer sheath 95 and the second outer sheath 94 are different sheaths and are arranged side by side to be adjacent to each other in the longitudinal direction L. The first outer sheath 95 is provided outside the outer cylinder 9 on the proximal side of the forceps port 232. The first outer sheath 95 is disposed adjacent to the outer peripheral surface of the outer cylinder 9 from the proximal end 92 of the outer cylinder 9 to the vicinity of the forceps port 232. The second outer sheath 94 is provided on the outer periphery of the adjuster 6 and the reinforcing portion 921. The first outer sheath 95 and the second outer sheath 94 may be an integrated sheath.

Since the delivery device 1E includes the reinforcing portion 921 between the adjuster 6 and the housing 30, when the treatment tool body 10 is bent at the time of placing the stent, the contact portion between the proximal end of the treatment tool body 10 and the housing 30 can withstand the force applied thereto due to the compressive force applied to the outer cylinder 9, and buckling and deformation of the treatment tool body 10 can be prevented. As a result, the outer cylinder 9 can be prevented from buckling and deforming due to the compressive force generated at the proximal end of the outer cylinder 9 at the time of placing the stent. Since the first outer sheath 95 and the second outer sheath 94 are provided, the rigidity of the treatment tool body 10 exposed on the proximal side of the forceps port 232 can be increased, and the effect of the straightening force applied to the outer cylinder 9 in a bent state can be reduced. Thus, as in the delivery device 1 of the first embodiment, the adjuster 6 can correct the relative change in path length, and the change in path length can be suppressed by preventing the straightening of the outer cylinder 9. As a result, since unintended advancing movement of the inner cylinder 8 is suppressed, the procedure can be performed smoothly, and the procedure time can be prevented from being prolonged.

### Fourth Embodiment

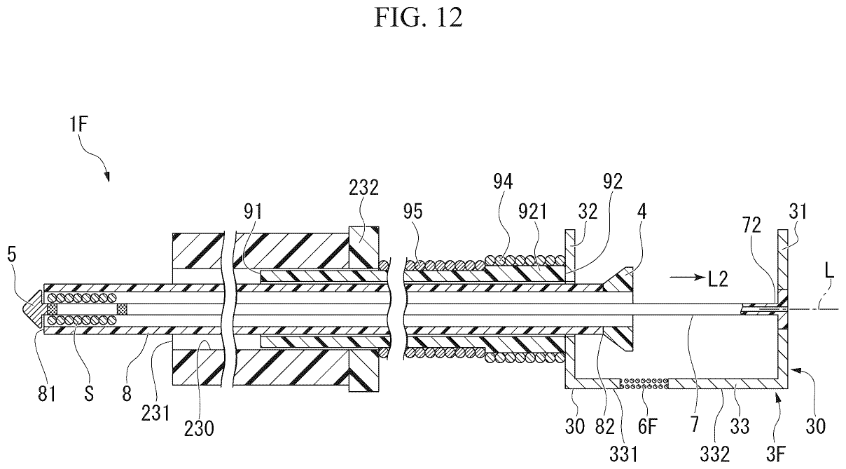

A delivery device 1F according to a fourth embodiment will be described with reference to FIG. 12. FIG. 12 is a cross-sectional view of the delivery device 1F according to this embodiment. The delivery device 1F includes a reinforcing portion 921 at the proximal end of the outer cylinder 9 to be thicker than the distal side. Further, the delivery device includes the first outer sheath 95 and the second outer sheath 94 outside the outer cylinder 9 on the proximal side of the forceps port 232. The other configurations are the same as those of the delivery device 1D of the second embodiment. Since the reinforcing portion 921 is provided in the outer cylinder 9, it is possible to prevent the outer cylinder 9 from buckling and deforming due to the compressive force generated at the proximal end of the outer cylinder 9 at the time of placing the stent. Since the first outer sheath 95 and the second outer sheath 94 are provided, the rigidity of the treatment tool body 10 exposed on the proximal side of the forceps port 232 can be increased, and the effect of the straightening force applied to the outer cylinder 9 in a bent state can be reduced. Thus, as in the delivery device 1D of the second embodiment, an adjuster 6F can correct the change in path length, and the change in path length can be suppressed by preventing the straightening of the outer cylinder 9. As a result, since unintended advancing movement of the inner cylinder 8 is suppressed, the procedure can be performed smoothly, and the procedure time can be prevented from being prolonged.

### Fifth Embodiment

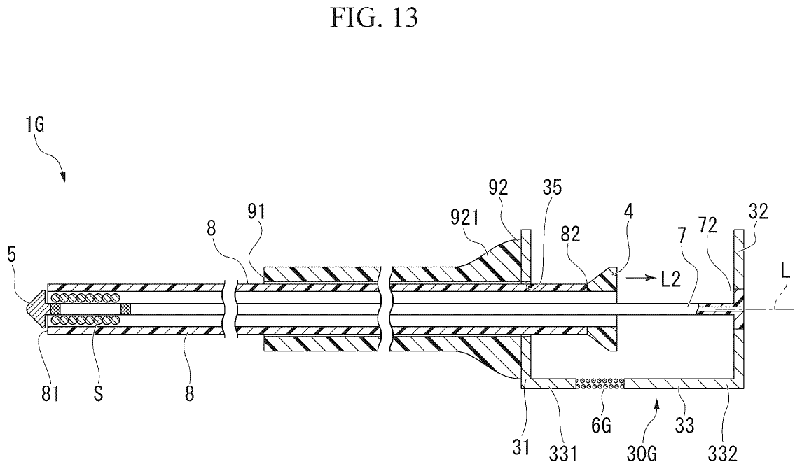

A delivery device 1G according to a fifth embodiment will be described with reference to FIG. 13. FIG. 13 is a cross-sectional view of the delivery device 1G according to this embodiment. The fifth embodiment is an example in which the reinforcing portion 921 is provided at the proximal end of the outer cylinder 9. The other configurations are the same as those of the delivery device 1D of the second embodiment. The reinforcing portion 921 is provided integrally with the outer cylinder 9. The reinforcing portion 921 is formed in a tapered shape so that a region of a part of the distal direction L1 from the proximal end 92 of the outer cylinder 9 becomes thicker toward the proximal end 92. In this way, since the reinforcing portion 921 is provided at the proximal end 92 of the outer cylinder 9, it is possible to prevent the outer cylinder 9 from buckling and deforming due to the compressive force generated at the proximal end of the outer cylinder 9 at the time of placing the stent. As a result, the procedure can be performed smoothly and the procedure time can be prevented from being prolonged.

According to the delivery devices 1, 1D, 1E, 1F, and 1G and the endoscope system 300 of the above-described embodiments, the stent S can be positioned at a desired position with high accuracy at the time of placing the stent.

According to the delivery devices 1, 1D, 1E, 1F, and 1G of the embodiments, since the outer cylinder 9 is provided outside the inner cylinder 8 and the shaft member 7 for placing the stent S, the rigidity of the treatment tool body 10 exposed on the proximal side of the forceps port 232 is increased. As a result, when the inner cylinder 8 is pulled in the proximal direction L2 to place the stent S, the straightening of the treatment tool body 10 can be prevented, and unintended advancing movement of the inner cylinder 8 can be suppressed. In addition, since the adjuster 6 is provided, it is possible to correct the relative position in the longitudinal direction L of the outer cylinder 9 relative to at least one of the inner cylinder 8 and the shaft member 7. That is, since the adjuster 6 is provided, it is possible to correct the compressive force generated in the outer cylinder 9 in accordance with the operation of pulling the inner cylinder 8 to place the stent S. Thus, unintended advancing movement of the inner cylinder 8 is prevented after the distal end of the treatment tool body 10 is aligned to the placement position T1 of the stent S. Further, it is possible to suppress a change in path length by eliminating the compressive force applied to the outer cylinder 9 when pulling the inner cylinder 8 to place the stent S. As a result, the stent S can be placed at a desired position with high accuracy.

According to the delivery device 1 of the embodiment, since the adjuster 6 is provided between the proximal end 92 of the outer cylinder 9 and the proximal end 72 of the shaft member 7, it is possible to correct the relative position between the proximal end 92 of the outer cylinder 9 and the proximal end 72 of the shaft member 7 in the longitudinal direction L at the time of pulling the inner cylinder 8. That is, since the adjuster 6 is provided, it is possible to correct the compressive force generated in the outer cylinder 9 in accordance with the operation of pulling the inner cylinder 8 to place the stent S between the proximal end 92 of the outer cylinder 9 and the proximal end 72 of the shaft member 7. Thus, unintended advancing movement of the inner cylinder 8 is prevented after the distal end of the treatment tool body 10 is aligned to the placement position T1 of the stent S. Further, it is possible to suppress a change in path length by eliminating the compressive force applied to the outer cylinder 9 when pulling the inner cylinder 8 to place the stent S. As a result, the stent S can be placed at a desired position with high accuracy.

According to the delivery device 1 of the embodiment, since the adjuster 6 is contractible between the reference length in the longitudinal direction L of the adjuster 6 when the outer cylinder 9 has a linear shape and no load is applied and the correction length which becomes shorter than the reference length due to an external force, the compressive force of the outer cylinder 9 can be corrected in the proximal direction L2.

According to the delivery device 1 of the embodiment, the proximal end 72 of the shaft member 7 is fixed to the housing 30 provided on the proximal side of the proximal end 92 of the outer cylinder 9, the proximal end 82 of the inner cylinder 8 is disposed inside the housing 30, and the adjuster 6 is provided between the proximal end 92 of the outer cylinder 9 and the proximal end 72 of the shaft member 7. Therefore, when the inner cylinder 8 is pulled, the compressive force generated in the outer cylinder 9 can be corrected in the vicinity of the operation section 3 by the adjuster 6. Therefore, it is possible to eliminate a change in path length on the hand side without changing the position of the distal end of the treatment tool body 10 after positioning the distal end of the treatment tool body 10 to the placement position T1.

According to the delivery device 1 of the embodiment, since the adjuster 6 is provided between the proximal end 92 of the outer cylinder 9 and the proximal end of the shaft member 7, it is possible to correct the compressive force generated in the outer cylinder 9 on the side of the proximal end 92 of the outer cylinder 9 by the adjuster 6 at the time of pulling the inner cylinder 8. Therefore, it is possible to eliminate a change in path length on the hand side without changing the position of the distal end of the treatment tool body 10 after positioning the distal end of the treatment tool body 10 to the placement position T1.

According to the delivery device 1 of the embodiment, since the adjuster 6 is provided in the housing 30, it is possible to prevent the housing 30 from being bent due to the pulling operation of the inner cylinder 8 and to perform a smooth operation.

According to the endoscope system 300 of the embodiment, since the delivery device 1 is provided, the stent S can be positioned at a desired position with high accuracy at the time of placing the stent. In addition, the endoscope system 300 with excellent operability can be provided.

In the above-described embodiments, an example in which the adjuster 6 is a coil spring has been shown, but the configuration of the adjuster is not limited to the coil spring. The adjuster 6 may be configured to include an elastic member that can contract in the longitudinal direction L when an external force is applied. For example, the adjuster 6 may be made of a resin tube having a soft portion with a lower hardness than the outer cylinder 9 in at least a part in the longitudinal direction L, and may be connected to the proximal end 92 of the outer cylinder 9. For example, the adjuster 6 may have a telescopic structure and a biasing member that biases in the expansion direction, and may be configured to contract in the longitudinal direction L when an external force is applied.

There are no particular limitations on the materials for the outer cylinder 9, the inner cylinder 8, the shaft member 7, and the housing 30, so long as the desired mechanical properties are satisfied. In the case of the delivery device used under X-ray fluoroscopy, radiopaque metallic markers (e.g., medical grade radiopaque metals and alloys such as platinum, tungsten, iridium, etc.) may be added or radiopaque materials (e.g., barium sulfate, etc.) may be mixed.

While the first embodiment has been described above in detail with reference to the drawings, the specific configuration is not limited to this embodiment, and design modifications and the like are also included within the scope of the present invention that does not depart from the gist of the present invention. Furthermore, the components shown in the above-described embodiment and the modified examples shown above can be combined as appropriate.

The present invention can be applied to a delivery device that delivers a medical tool into a body.

What is claimed is:

1. A delivery device comprising:
an outer cylinder;
an inner cylinder which is configured to be inserted through the outer cylinder;
a shaft member that is a flexible elongated member and is configured to be inserted through the inner cylinder to hold a stent between a distal end portion and the inner cylinder;
an operation mechanism which includes a housing and is operated to relatively move the inner cylinder relative to the shaft member in a longitudinal direction; and
an adjuster which is provided from a proximal end of the outer cylinder to a proximal end of the shaft member and is configured to correct the relative position of the outer cylinder relative to at least one of the inner cylinder or the shaft member in the longitudinal direction.

2. The delivery device according to claim 1,
wherein the adjuster is configured to include an elastic member that is expandable and contractable in the longitudinal direction, and
wherein the adjuster is expandable and contractible between a reference length in the longitudinal direction of the adjuster when the outer cylinder has a linear shape and no load is applied and a correction length which becomes shorter than the reference length due to an external force.

3. The delivery device according to claim 1,
wherein the adjuster contracts in the longitudinal direction by receiving a force in a proximal side from the outer cylinder.

4. The delivery device according to claim 1,
wherein the operation mechanism includes a handle which is provided at a proximal end of the inner cylinder and is operated to advance and retract inside the housing so that the inner cylinder is operated to advance and retract relative to the shaft member, and
wherein a proximal end of the shaft member is fixed to the housing.

5. The delivery device according to claim 1,
wherein the adjuster is provided in the housing.

6. The delivery device according to claim 1,
wherein the adjuster is provided between a proximal end of the outer cylinder and the housing.

7. A delivery device comprising:
an outer cylinder;
an inner cylinder which is configured to be inserted through the outer cylinder;
a shaft member that is a flexible elongated member and is configured to be inserted through the inner cylinder to hold a stent between a distal end portion and the inner cylinder;
an operation mechanism which includes a housing and is operated to relatively move the inner cylinder relative to the shaft member in a longitudinal direction; and
an adjuster which is provided from a proximal end of the outer cylinder to a proximal end of the shaft member and is configured to correct the relative position between a proximal end of the outer cylinder and a proximal end of the shaft member in the longitudinal direction.

8. The delivery device according to claim 7,
wherein the adjuster is configured to include an elastic member that is expandable and contractable in the longitudinal direction, and
wherein the adjuster is expandable and contractible between a reference length in the longitudinal direction of the adjuster when the outer cylinder has a linear shape and no load is applied and a correction length which becomes shorter than the reference length due to an external force.

9. The delivery device according to claim 7,
wherein the adjuster is configured to include an elastic member that is expandable and contractable in the longitudinal direction, and
wherein the adjuster is expandable and contractible between a reference length in the longitudinal direction of the adjuster when the outer cylinder has a linear shape and no load is applied and a correction length which becomes shorter than the reference length due to an external force.

10. The delivery device according to claim 7,
wherein the adjuster contracts in the longitudinal direction by receiving a force in a proximal side from the outer cylinder.

11. The delivery device according to claim 7,
wherein the operation mechanism includes a handle which is provided at a proximal end of the inner cylinder and is operated to advance and retract inside the housing so that the inner cylinder is operated to advance and retract relative to the shaft member, and
wherein a proximal end of the shaft member is fixed to the housing.

12. The delivery device according to claim 7,
wherein the adjuster is provided in the housing.
